(19) United States
(12) Patent Application Publication
(10) Pub. No.: US 2025/0295348 A1
(43) Pub. Date: Sep. 25, 2025

(54) METHODS AND SYSTEMS FOR OPTIMIZING AMBULATORY ECG SIGNAL COMPRESSION AND RECONSTRUCTION

(71) Applicant: KONINKLIJKE PHILIPS N.V., EINDHOVEN (NL)

(72) Inventors: Joshua Michael Burns, San Diego, CA (US); Justin Tsoneshane Lee, San Diego, CA (US)

(21) Appl. No.: 19/089,077

(22) Filed: Mar. 25, 2025

Related U.S. Application Data
(60) Provisional application No. 63/569,366, filed on Mar. 25, 2024.

(51) Int. Cl. A61B 5/349; A61B 5/00

(52) U.S. Cl. CPC A61B 5/349; A61B 5/0006; A61B 5/7232; A61B 5/7264; A61B 5/742

(57) ABSTRACT

A method for compressing and transmitting an ECG signal by an ambulatory electrocardiogram (ECG) device, comprising: obtaining an ECG signal from a subject; determining whether the obtained ECG signal comprises ECG beat locations; pre-processing the obtained ECG signal with a de-trending analysis to generate a pre-processed ECG signal if the obtained ECG signal is determined to comprise ECG beat locations, or pre-processing the obtained ECG signal with a bandpass filter to generate a pre-processed ECG signal if the obtained ECG signal is determined to not comprise ECG beat locations; approximating the pre-processed ECG signal to generate an approximated ECG signal; quantizing the approximated ECG signal to generate a quantized ECG signal; generating a compressed ECG signal from the quantized ECG signal; transmitting the compressed ECG signal.

### FIELD OF THE DISCLOSURE

The present disclosure is directed generally to methods and systems for optimizing the compression and transmission of an electrocardiogram (ECG) signal by an ambulatory ECG device.

### BACKGROUND

The electrocardiogram (ECG) is a non-invasive technique that measures the electrical activity of the heart and is used to detect, among other things, cardiac disorders. Most commonly, ECG measurements are collected in a hospital or in an ambulatory setting. Ambulatory settings typically utilize either a Holter® monitor or mobile cardiac telemetry (MCT).

During ambulatory monitoring, the Holter monitor requires that ECG data be collected and stored on the monitor, while MCT processes the ECG data in semi-real time and sends ECG events based on classification. Both approaches have advantages and disadvantages.

Holter and MCT devices each have their advantages and disadvantages. Holter devices are easier for a patient and physician to apply and maintain, provide the full ECG data for review, and allow for more accurate algorithms to classify arrhythmias. However, for analysis, the patient and/or physician must either mail back the device or upload the data to a server. Deep learning algorithms are often used for the analysis of ECG data obtained by a Holter monitor. These algorithms cannot be implemented on the device because they consume too much computing power and battery life. For offline processing, there is a delay in treatment because you must wait for the mail back of the device and uploading of the data. This results in delayed diagnosis which may be critical if the patient needs hospitalization.

MCT devices analyze the ECG data in semi-real time and transmits ECG data that have been identified as important events. MCT monitoring has the disadvantage that the full ECG data is not available, only the important events that have been transmitted. In addition, algorithms that use large amounts of computer processing or memory may not be viable on the device due to resources and/or consumption of battery life.

### SUMMARY OF THE DISCLOSURE

There is thus a continued need for methods and systems that optimize the compression and transmission of an electrocardiogram (ECG) signal by an ambulatory ECG device, such that an entire ECG signal is transmitted and signal fidelity is maintained.

Various embodiments and implementations are directed to a method and system for the optimized compression of an ECG signal by an ambulatory ECG device, and the subsequent and reconstruction of the transmitted ECG signal. The ambulatory ECG device obtains an ECG signal from a subject and processes the ECG signal to generate a compressed ECG signal that can be transmitted in full. ECG data compression reduces the number of bits needed to represent the ECG data, which saves storage capacity, speeds up file transfer, and decreases costs for storage hardware and network bandwidth. The ambulatory ECG devices and methods disclosed or otherwise envisioned herein maintain the advantages of traditional ambulatory ECG analysis while avoiding the disadvantages of these prior art methods by compressing and reconstructing the ECG signal in way that the signal fidelity is maintained. Once the compressed ECG data is transmitted, it can be processed using ECG classification algorithms to generate a reconstructed ECG signal.

According to an aspect, a method for compressing and transmitting an ECG signal by an ambulatory ECG device is provided. The method includes: (i) obtaining, by the ambulatory ECG device, an ECG signal from a subject; (ii) determining, by a processor of the ambulatory ECG device, whether the obtained ECG signal comprises ECG beat locations; (iii) pre-processing, by the processor, the obtained ECG signal with a de-trending analysis to generate a pre-processed ECG signal if the obtained ECG signal is determined to comprise ECG beat locations, or pre-processing, by the processor, the obtained ECG signal with a bandpass filter to generate a pre-processed ECG signal if the obtained ECG signal is determined to not comprise ECG beat locations; (iv) approximating, by the processor, the pre-processed ECG signal to generate an approximated ECG signal; (v) quantizing, by the processor, the approximated ECG signal to generate a quantized ECG signal; (vi) generating, by a compression algorithm of the processor, a compressed ECG signal from the quantized ECG signal; (vii) transmitting, by the ambulatory ECG device, the compressed ECG signal.

According to an embodiment, the method further includes: receiving, the transmitted compressed ECG signal; reconstructing, by a reconstruction algorithm, the compressed ECG signal to generate a reconstructed approximated ECG signal; and transforming, by a trained neural network, the reconstructed approximated ECG signal to generate a decompressed ECG signal.

According to an embodiment, the method further includes displaying at least a portion of the decompressed ECG signal.

According to an embodiment, the transmitted compressed ECG signal is received, reconstructed, and analyzed by a server remote from the ambulatory ECG device.

According to an embodiment, approximating the pre-processed ECG signal to generate an approximated ECG signal comprises: (i) a wavelet transform; (ii) a selection of largest wavelet coefficients; and (iii) a tolerance determination.

According to an embodiment, a one-second windowed ECG signal around each beat in at least a portion of the obtained ECG signal is utilized to determine the tolerance.

According to an embodiment, quantizing the approximated ECG signal to generate a quantized ECG signal comprises quantizing wavelet coefficients of the ECG signal using a quantization parameter (A).

According to an embodiment, generating a compressed ECG signal from the quantized ECG signal comprises saving the compressed ECG signal as an HDF5 file.

According to an embodiment, the compressed ECG signal further comprises encoded metadata.

According to another aspect of the invention is an ambulatory electrocardiogram (ECG) device. The device includes: one or more ECG leads configured to obtain an ECG signal from a subject; a processor configured to: (i) determine whether the obtained ECG signal comprises ECG beat locations; (ii) pre-process the obtained ECG signal with a de-trending analysis to generate a pre-processed ECG signal if the obtained ECG signal is determined to comprise ECG beat locations, or pre-process the obtained ECG signal with a bandpass filter to generate a pre-processed ECG signal if the obtained ECG signal is determined to not comprise ECG beat locations; (iii) approximate, by the processor, the pre-processed ECG signal to generate an approximated ECG signal; (iv) quantize the approximated ECG signal to generate a quantized ECG signal; and (v) generate a compressed ECG signal from the quantized ECG signal; and a communications interface configured to transmit the compressed ECG signal.

According to another aspect of the invention is an electrocardiogram (ECG) system. The system includes an ambulatory ECG device comprising: one or more ECG leads configured to obtain an ECG signal from a subject; a processor configured to: (i) determine whether the obtained ECG signal comprises ECG beat locations; (ii) pre-process the obtained ECG signal with a de-trending analysis to generate a pre-processed ECG signal if the obtained ECG signal is determined to comprise ECG beat locations, or pre-process the obtained ECG signal with a bandpass filter to generate a pre-processed ECG signal if the obtained ECG signal is determined to not comprise ECG beat locations; (iii) approximate, by the processor, the pre-processed ECG signal to generate an approximated ECG signal; (iv) quantize the approximated ECG signal to generate a quantized ECG signal; and (v) generate a compressed ECG signal from the quantized ECG signal; and a communications interface configured to transmit the compressed ECG signal. The ECG system also includes a remote server comprising: a communications interface configured to receive the transmitted compressed ECG signal; and a processor configured to: (i) reconstruct the compressed ECG signal to generate a reconstructed approximated ECG signal; and (ii) transform, by a trained neural network, the reconstructed approximated ECG signal to generate a decompressed ECG signal.

According to an embodiment, the remote server further comprises a user interface configured to display at least a portion of the decompressed ECG signal.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

These and other aspects of the various embodiments will be apparent from and elucidated with reference to the embodiment(s) described hereinafter.

### BRIEF DESCRIPTION OF THE DRAWINGS

In the drawings, like reference characters generally refer to the same parts throughout the different views. The figures showing features and ways of implementing various embodiments and are not to be construed as being limiting to other possible embodiments falling within the scope of the attached claims. Also, the drawings are not necessarily to scale, emphasis instead generally being placed upon illustrating the principles of the various embodiments.

FIG. 1A is a flowchart of a method for compressing and transmitting an electrocardiogram (ECG) signal by an ambulatory ECG device, in accordance with an embodiment.

FIG. 1B is a flowchart of a method for reconstructing an ECG signal, in accordance with an embodiment.

FIG. 2 is a schematic representation of an ECG device, in accordance with an embodiment.

FIG. 3 is a schematic representation of an ECG system, in accordance with an embodiment.

FIG. 4 is a flowchart of a method for pre-processing an obtained ECG signal with a de-trending analysis to generate a pre-processed ECG signal, in accordance with an embodiment.

FIG. 5 is a flowchart of a method for generating an approximated ECG signal, in accordance with an embodiment.

Referring to FIG. 6, in one embodiment, is a flowchart of a method for quantizing wavelet coefficients, in accordance with an embodiment.

Referring to FIG. 7, in one embodiment, is a flowchart of a method for organizing and saving an ECG signal as a compressed ECG signal, in accordance with an embodiment.

Referring to FIG. 8, in one embodiment, is a flowchart of a method for breaking down vi into two arrays, in accordance with an embodiment.

Referring to FIG. 9, in one embodiment, is a flowchart of a method for generating a reconstructed approximated ECG signal, in accordance with an embodiment.

Referring to FIG. 10, in one embodiment, is a schematic representation of a neural network autoencoder 1000, in accordance with an embodiment.

Referring to FIG. 11, in one embodiment, is a flowchart of a method for training a neural network autoencoder of the ECG system, in accordance with an embodiment.

Referring to FIG. 12, in one embodiment, is a graph of a single heartbeat from an ECG signal, in accordance with an embodiment.

### DETAILED DESCRIPTION OF EMBODIMENTS

The present disclosure describes various embodiments of a system and method configured to optimize the compression and transmission of an electrocardiogram (ECG) signal by an ambulatory ECG device. More generally, Applicant has recognized and appreciated that it would be beneficial to provide a method and system to overcome the limitations of ECG data transmission and analysis by existing ambulatory ECG devices, such that an entire ECG signal is transmitted and signal fidelity is maintained. The ambulatory ECG device obtains an ECG signal from a subject and processes the ECG signal to generate a compressed ECG signal that can be transmitted in full. ECG data compression reduces the number of bits needed to represent the ECG data, which saves storage capacity, speeds up file transfer, and decreases costs for storage hardware and network bandwidth. The ambulatory ECG devices and methods disclosed or otherwise envisioned herein maintain the advantages of traditional ambulatory ECG analysis while avoiding the disadvantages of these prior art methods by compressing and reconstructing the ECG signal in way that the signal fidelity is maintained. Once the compressed ECG data is transmitted, it can be processed using ECG classification algorithms to generate a reconstructed ECG signal.

The embodiments and implementations disclosed or otherwise envisioned herein can be utilized with any ECG system or process that may utilize or benefit from analysis of high fidelity ECG signals obtained from a subject. The embodiments and implementations disclosed or otherwise envisioned herein can be utilized with any system that obtains ECG data, including but not limited to ambulatory ECG devices and systems, among other products. However, the disclosure is not limited to these devices or systems, and thus disclosure and embodiments disclosed herein can encompass any system that may utilize or benefit from analysis of high fidelity ECG signals obtained from a subject.

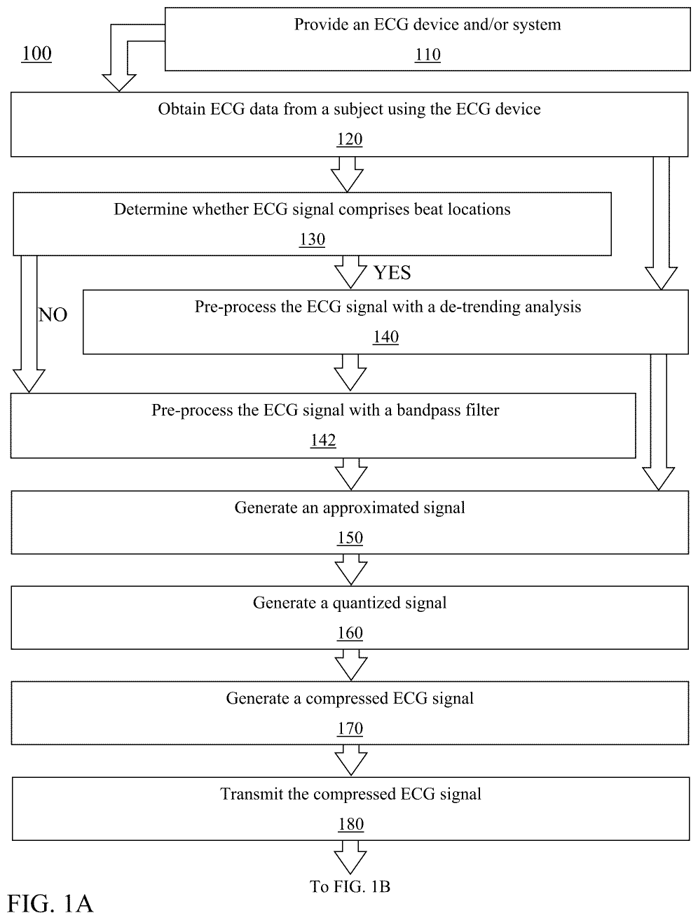

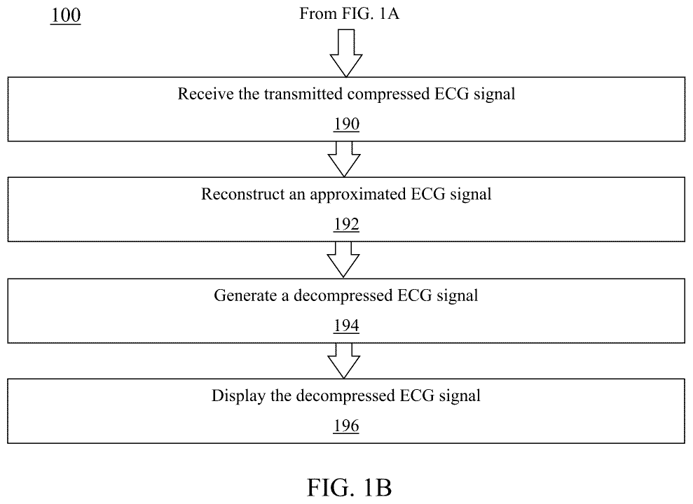

Referring to FIG. 1A, in one embodiment, is a flowchart of a method 100 for optimizing the compression and transmission of an ECG signal using an ambulatory ECG device or system. The methods described in connection with the figures are provided as examples only, and shall be understood not to limit the scope of the disclosure. The ECG device or system can be any of the devices or systems described or otherwise envisioned herein. The ECG device or system can be a single device or system, or can be multiple different devices or systems.

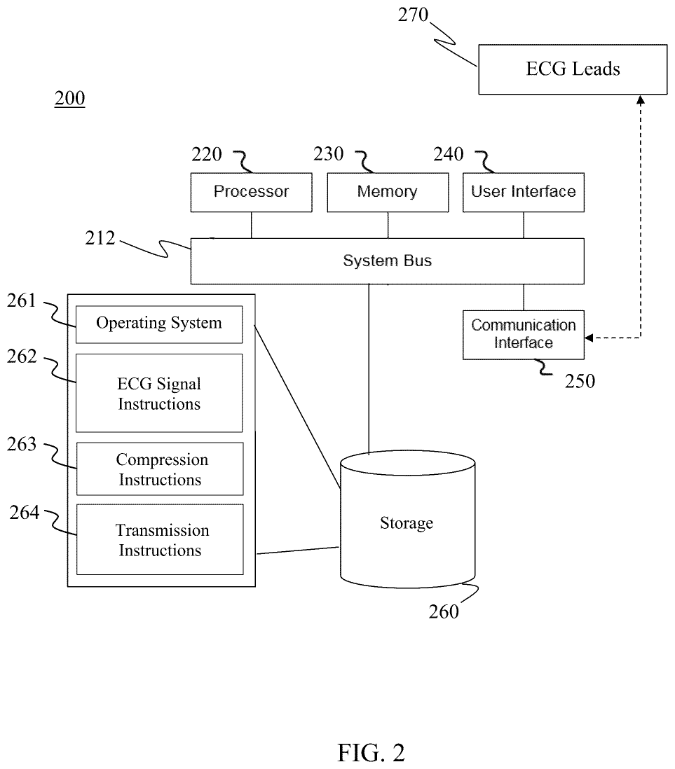

At step 110 of the method, an ECG device 200 or ECG system 300 is provided. Referring to an embodiment of an ECG device 200 as depicted in FIG. 2, for example, the system comprises one or more of a processor 220, memory 230, user interface 240, communications interface 250, and storage 260, interconnected via one or more system buses 212. It will be understood that FIG. 2 constitutes, in some respects, an abstraction and that the actual organization of the components of the ECG device 200 may be different and more complex than illustrated. Additionally, ECG device 200 can be any of the devices described or otherwise envisioned herein. Other elements and components of the ECG device 200 are disclosed and/or envisioned elsewhere herein.

According to an embodiment, the ECG device 200 comprises or is in direct or indirect communication with one or more ECG leads 270 configured to obtain ECG data from a subject. Accordingly, the one or more ECG leads 270 will be positioned in contact with the subject, according to known methods, and can obtain ECG data from that subject. Once obtained, the ECG data may be utilized immediately, and/or it may be temporarily or permanently stored in memory for future use.

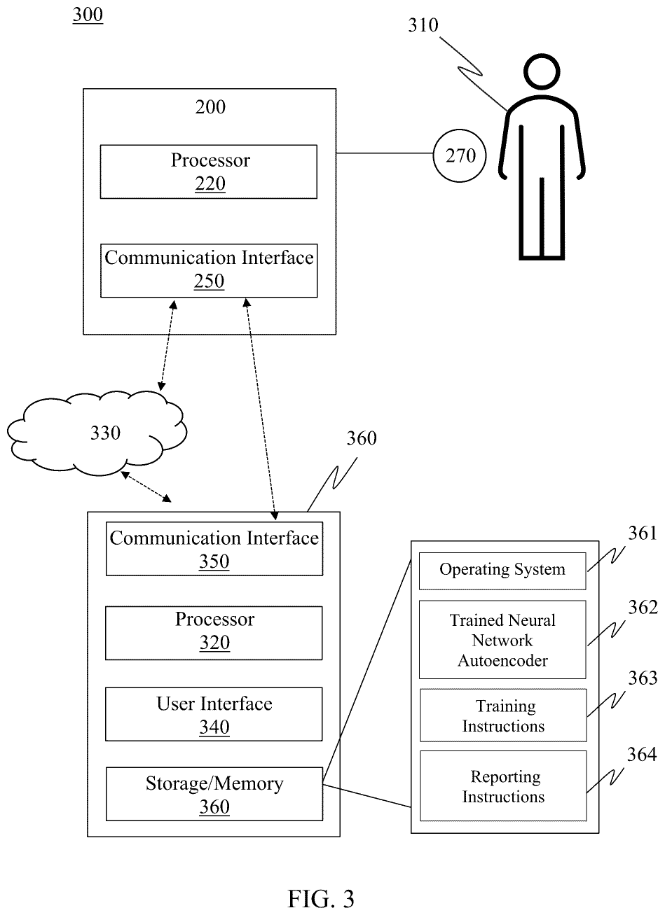

Additionally, or alternatively, at step 110 of the method, an ECG system 300 is provided. Referring to an embodiment of an ECG system 300 as depicted in FIG. 3, for example, the system comprises one or more of an ECG device 200, which according to an embodiment is an ambulatory ECG device, which comprises one or more ECG leads 270 configured to obtain an ECG signal from a subject 310. System 300 further comprises a remote server or computer 360, which receives a compressed ECG signal from the ECG device 200. For example, the compressed ECG signal can be transmitted by the communication interface 250 of the ECG device to the communication interface 350 of the remote server or computer 360, either directly or through a communications network 330. Once received, the compressed ECG signal can be stored in memory, and/or can be analyzed by a processor 320 of the remote server or computer 360 to generate a reconstructed ECG signal. The remote server or computer 360 further comprises or is in direct or indirect communication with a user interface 340 that can be utilized to display one or more aspects of a reconstructed ECG signal.

At step 120 of the method, the ECG device 200 obtains an ECG signal from a subject 310 using the one or more ECG leads 270. The ECG device may obtain an ECG signal using known methods for obtaining ECG signals. The signal may be obtained continuously or periodically as determined by the settings or parameters of the ECG device or system. When the ECG device is an ambulatory device, the ECG signal may be obtained while the subject is ambulatory. Thus, according to an embodiment, “ambulatory” can mean that the subject is utilizing a mobile ECG device outside a clinical setting, rather than using a stationary or mobile ECG device within a clinical setting. Once obtained, the ECG signal may be utilized immediately and/or it may be saved in memory for future analysis.

At step 130 of the method, the processor 220 of the ECG device 200 analyzes the obtained ECG signal to determine whether the signal comprises ECG beat locations. According to an embodiment, beat locations refers to specific points on the ECG waveform (i.e., the representation of the electrical activity of the heart from the obtained ECG signal) that represent the sequence of electrical events during a single heartbeat. Identifying and analyzing these beat locations is crucial for diagnosing cardiac abnormalities, arrhythmias, and other heart conditions. An ECG signal might not include identifiable beat locations for several reasons, often related to technical issues, patient-specific factors, or pathological conditions affecting the heart's electrical activity. The outcome of the analysis of processor 220 of ECG device 200 is a determine that either: (1) the obtained ECG signal comprises ECG beat locations; or (2) the obtained ECG signal does not comprise ECG beat locations. According to an embodiment, the determination may be based in whole or in part on a threshold such that there may be some, but not enough, beat locations in the ECG signal. The threshold may be determined experimentally and/or it may be manually determined or set.

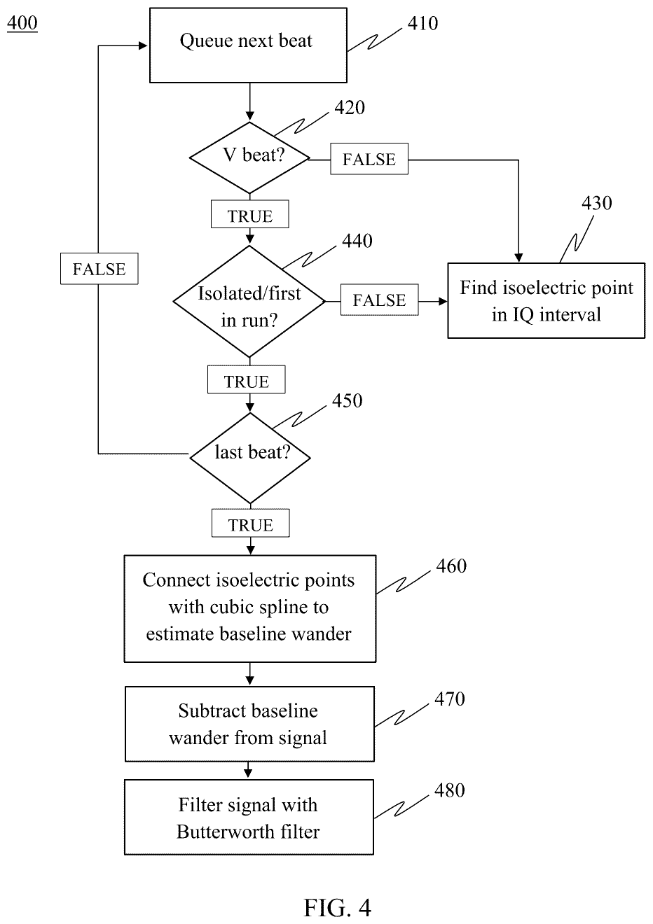

When the ECG device determines that the obtained ECG signal comprises ECG beat locations, then at step 140 of the method the processor 220 of ECG device 200 pre-processes the obtained ECG signal with a de-trending analysis to generate a pre-processed ECG signal. The de-trending analysis may be performed using a variety of different methods. Referring to FIG. 4, in one embodiment, is a flowchart of a method 400 for pre-processing an obtained ECG signal with a de-trending analysis to generate a pre-processed ECG signal. Method 400 is provided as one possible de-trending analysis, and thus should be understood not to limit the scope of the disclosure.

According to an embodiment, the ECG signal is detrended of baseline wander and denoised prior to the application of the compression algorithm. It is critical to pre-process the signal before compression because the signal reconstruction following compression/decompression will lose important information if the original signal contains large amounts of low/high frequency noise. Thus, according to an embodiment, a baseline wander removal algorithm subtracts an estimate of the baseline wander as generated by PQ-segment isoelectric amplitudes. For isolated ventricular beats or the first in a run of V beats, the method is applied. Even though V beats do not have a P-wave, the isoelectric point identification method will place the fiducial marker directly before the V beat. This method is not applied to subsequent beats in a V beat run because the fiducial point marked can be the valley of the T-wave or S-wave of the prior V beat. An estimate of the baseline wander is determined by connecting a cubic spline across all the fiducial points found throughout the strip. This baseline wander estimate is then subtracted from the original signal to get a detrended ECG strip. According to an embodiment, the output of the baseline wander removal algorithm is then filtered using an 8-tap lowpass Butterworth filter with a cutoff frequency of 40 Hz. This reduces some high frequency noise before the application of the compression algorithm.

According to an embodiment, referring to method 400 in FIG. 4, at step 410 of the method the system queues the next beat in an ECG signal. The system determines at step 420 whether the beat is a V beat (i.e., a ventricular ectopic beat, also known as a ventricular premature beat (VPB) or premature ventricular contraction (PVC)). If no (“false” in FIG. 4), then the system finds the isoelectric point in the IQ interval at step 430. If the beat is a V beat (“true” in FIG. 4), then the system determines at step 440 whether the beat is isolated or first in a run; if no (“false” in FIG. 4), then the system finds the isoelectric point in the IQ interval at step 430. If yes (“true” in FIG. 4), then the system determines whether the beat is the last beat at step 450. If no (“false” in FIG. 4), then the system returns to step 410 to queue the next beat. If yes (“true” in FIG. 4), then at step 460 the system utilizes the obtained isoelectric points (from step 430).

Specifically, at step 460 of method 400, the system combines the obtained isoelectric points with a cubic spline to estimate baseline wander. Thus, an estimate of the baseline wander is determined by connecting a cubic spline across all the fiducial points found throughout the ECG signal.

At step 470 of the method, the system subtracts the baseline wander estimate from the original ECG signal to get a detrended ECG signal.

According to an embodiment, at step 480 of the method, the system filters the detrended ECG signal using a Butterworth filter or another filter. According to one possible embodiment, the output of the baseline wander removal algorithm is filtered using an 8-tap lowpass Butterworth filter with a cutoff frequency of 40 Hz. This reduces high frequency noise before the application of the compression algorithm.

The output of the method, either the detrended ECG signal or the filtered detrended ECG signal, can be utilized immediately and/or it may be stored in memory for future use.

Returning to method 100 in FIG. 1A, when the ECG device determines that the obtained ECG signal does not comprise ECG beat locations, then at step 142 of the method the processor 220 of ECG device 200 pre-processes the obtained ECG signal with a bandpass filter to generate a pre-processed ECG signal. Once filtered, the pre-processed ECG signal can be utilized immediately and/or it may be stored in memory for future use.

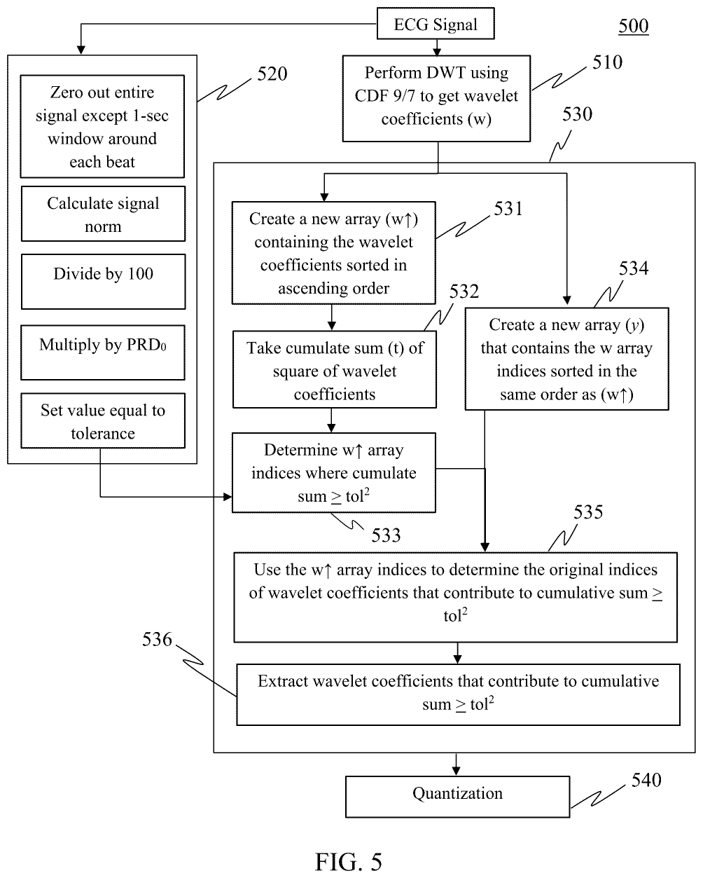

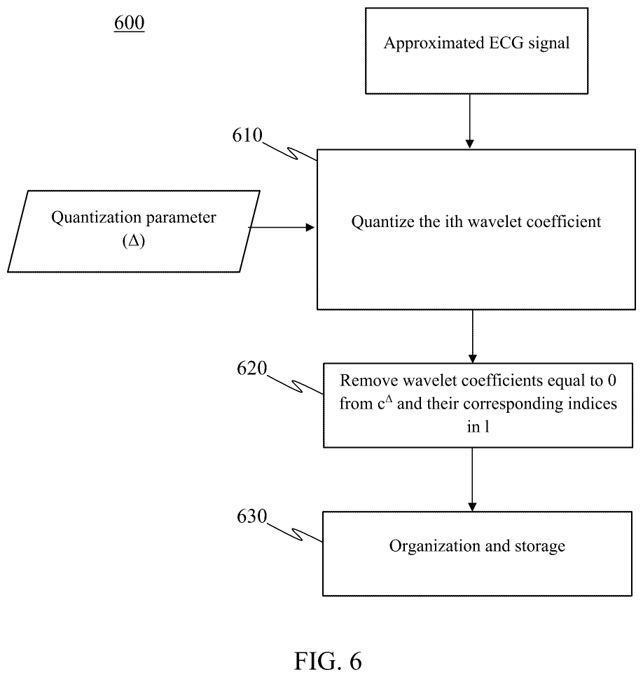

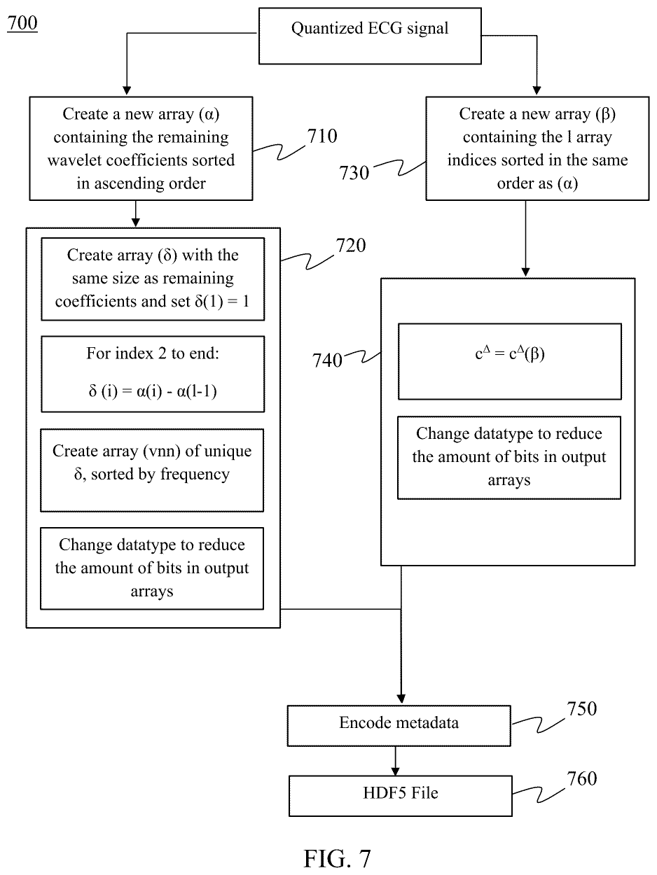

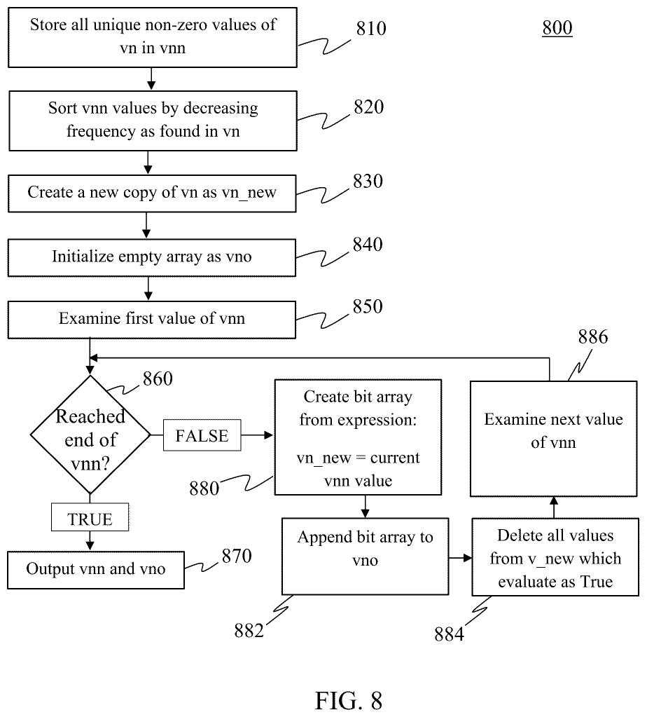

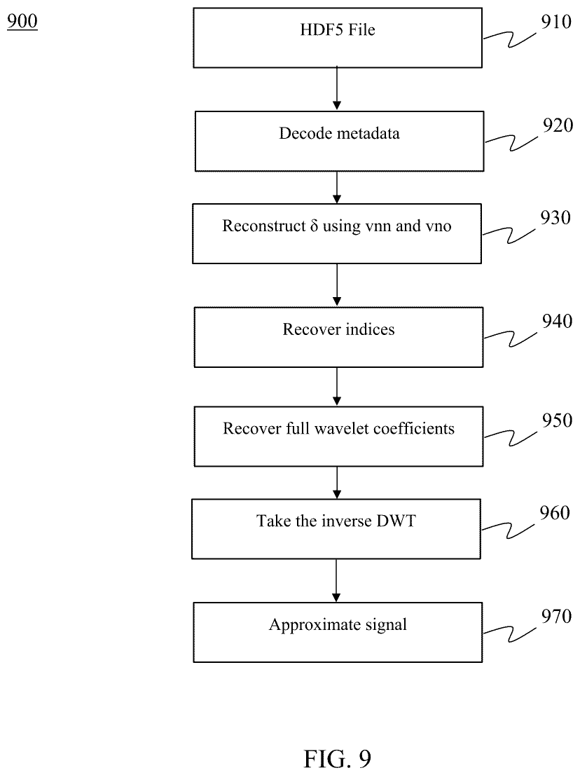

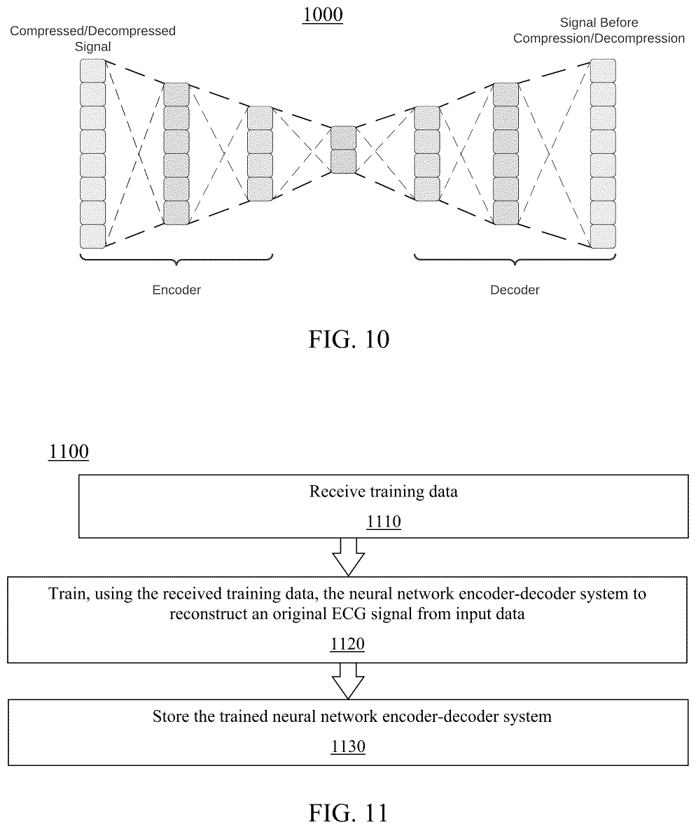

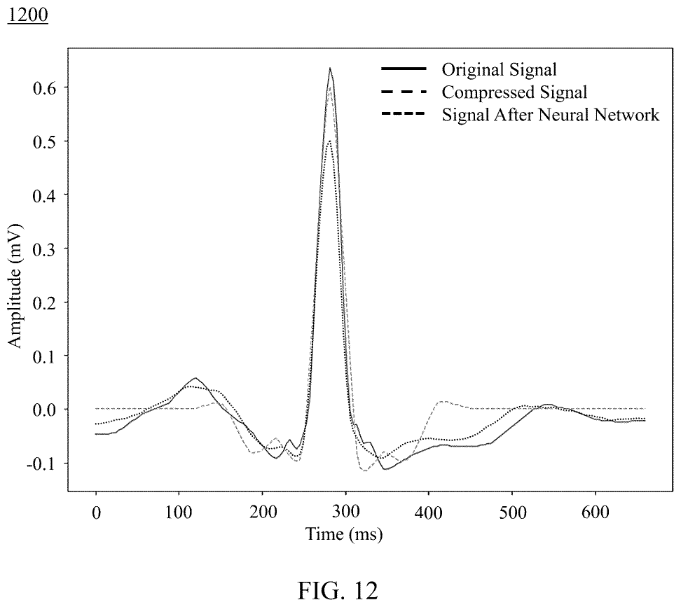

At step 150 of the method, the pre-processed ECG signal is analyzed by the processor 220 of the ECG device 200 to generate an approximated ECG signal. The analysis may be performed using a variety of different methods. Referring to FIG. 5, in one embodiment, is a flowchart of a method 500 for generating an approximated ECG signal. Method 500 is provided as one possible analysis, and thus should be understood not to limit the scope of the disclosure.

According to an embodiment, before processing applying the compression algorithm, the signal is clipped to lie between 2047 and −2048 ADC values if it is in MIT 212 format. The approximation step can be broken down into three main components: (1) wavelet transform; (2) selection of largest coefficients; and (3) tolerance determination. According to an embodiment, a one-second windowed ECG signal around each beat (determined by annotations) can be used to calculate the tolerance in order to mitigate the influence of high amplitude artifacts.

According to an embodiment, referring to method 500 in FIG. 5, at step 510 of the method the system performs a wavelet transform. According to an embodiment, the wavelet transform is performed by Discrete Wavelet Transform (DWT) using a Cohen-Daubechies-Feauveau wavelet transform (where 9/7 indicates the number of coefficients in the decomposition and reconstruction filters), to obtain the wavelet coefficients (w), although other methods and parameters are possible. Thus, according to an embodiment, the system decompose the ECG signal into detailed components using a specific wavelet transform and then uses dynamic time warping to analyze or compare these components. This approach can enhance the detection and interpretation of complex ECG features, potentially leading to more accurate diagnosis of cardiac conditions.

At step 520 of the method, the system determines tolerance. The tolerance can be determined using a variety of mechanisms. According to the embodiment depicted in FIG. 5, the system zeros out the entire signal except for a window around each beat. The window can be a variety of different time periods around a beat. According to one embodiment, for example, the window is 1-second around the beat, although many other windows are possible. The system calculates the signal norm, divides that by 100, and multiplies by the Percentage Root-mean-square Difference (PRD₀). The system then sets a value equal to the determined tolerance.

At step 530 of the method, the system selects the largest coefficients. The largest coefficients can be selected using a variety of mechanisms. According to the embodiment depicted in FIG. 5, at step 531 the system creates a new array (w^{†}) containing the obtained wavelet coefficients (w) sorted in ascending order. At step 532, the system takes the cumulate sum (t) of the square of the wavelet coefficients, using the equation:

$$t = cumsum ( {w}^{↑ 2} ) \qquad ( Eq . 1 )$$

At step 533, the system determines the new array (w^{†}) indices where the cumulative sum is greater than or equal to the square of the tolerance (tol²) (as determined in step 520 of the method).

At step 534, the system creates a new array (y) that contains the w array indices sorted in the same order as (w^{†}).

Then at step 535, the system uses the array (w^{†}) indices to determine the original indices of wavelet coefficients that contribute to cumulative sum greater than or equal to the square of the tolerance (tol²), using the equation:

$$1 = y ( array ( {w}^{↑} ) indices ) \qquad ( Eq . 2 )$$

At step 536, the system extracts wavelet coefficients that contribute to cumulative sum greater than or equal to the square of the tolerance (tol²) (as determined in step 520 of the method), using the equation:

$$c = w ( 1 ) \qquad ( Eq . 3 )$$

This results in an approximated ECG signal. The approximated ECG signal may be utilized immediately and/or may be stored in memory for future use.

Following the approximation, the wavelet coefficients are quantized and removed below the threshold. Thus, at step 540 of the method, the resulting wavelet coefficients are quantized and removed below the threshold to generate an approximated ECG signal.

Returning to method 100 depicted in FIG. 1A, at step 160 of the method, the approximated ECG signal is quantized to generate a quantized signal.

Referring to FIG. 6, in one embodiment, is a method 600 for quantizing the wavelet coefficients. The wavelet coefficients may be quantized using a variety of methods, including but not limited to the method depicted in FIG. 6. At step 610 of the method, the system receives the approximated ECG signal. The system quantizes the ith wavelet coefficient using a quantization parameter (Δ), which may be derived or determined using a variety of different mechanisms.

According to an embodiment, the system utilizes the following equation:

$${c}^{Δ} ( i ) = [ \frac{c ( i )}{Δ} + \frac{1}{2} ] \qquad ( Eq . 4 )$$

At step 620 of the method, the system removes the wavelet coefficients equal to 0 from c^{Δ} and their corresponding indices in 1.

This results in a quantized ECG signal. The quantized ECG signal may be utilized immediately and/or may be stored in memory for future use.

Following the quantizing, the signal can be organized and stored. Returning to method 100 depicted in FIG. 1A, at step 170 of the method, the quantized signal is compressed to generate a compressed ECG signal.

Referring to FIG. 7, in one embodiment, is a method for organizing and saving the ECG signal as a compressed ECG signal. The ECG signal may be organized and saved quantized using a variety of methods, including but not limited to the method depicted in FIG. 7.

At step 710 of the method, the system creates a new array (α) containing the remaining wavelet coefficients sorted in ascending order.

At step 720 of the method, the system stores the wavelet coefficient indices. According to an embodiment, the system creates a new array (δ) with the same size as the remaining coefficients and sets δ(1)=1. The system also sets for index 2 to end, the following:

$$δ ( i ) = α ( i ) - α ( 1 - 1 ) \qquad ( Eq . 5 )$$

The system creates an array (vnn) of unique δ, sorted by frequency. The system then changes the datatype to reduce the amount of bits in output arrays.

At step 730 of the method, the system creates a new array (β) containing the 1 array indices sorted in the same order as (δ). At step 740, the system stores the wavelet coefficient values, using:

$${c}^{Δ} = {c}^{Δ} ( β ) \qquad ( Eq . 6 )$$

The system then changes the datatype to reduce the amount of bits in output arrays.

At step 750 the system encodes metadata, and generates an HDF5 file, although other file formats are possible.

According to an embodiment, generating the compressed ECG file further comprises separating the wavelet coefficients into three different arrays: (1) vi which stores the wavelet coefficient indices; (2) vc which stores the amplitudes of the wavelet coefficient, and (3) vs which stores the signs of the of the wavelet coefficient. According to an embodiment, the system removes vs, retains the wavelet coefficient values in vc, and breaks down vi into two arrays. These changes further improve the compression level of the ECG signal. These arrays contain all of the compressed data to represent the ECG signal. They are embedded into the file (such as an HDF5 file) and the file is the compressed signal packet that gets sent back to the server. To decompress, the arrays are extracted from the file and used to recreate the wavelet coefficients.

Referring to FIG. 8, in one embodiment, is a flowchart of a method 800 for breaking down vi into two arrays. At step 810 of method 800, the system stores all unique non-zero values of vn in vnn. At step 820, these vnn values are sorted by decreasing frequency (as found in vn), and at 830 a new copy of vn is created as vn_new.

At step 840, the system initializes a new empty array as vno, and at step 850, the system examines the first value of vnn. If, at 860, the system has reached the end of vnn, then at 870 the system outputs vnn an vno. If, at 860, the system has not reached the end of vnn, then at 880 the system creates a bit array using the following equation:

$$vn\_new = current vnn value \qquad ( Eq . 7 )$$

The system then, at 882, the system appends the created bit array to vno, and at 884 deletes all values from v_new which evaluate as True. Then system then, at 886, examines the next value of vnn (if there is one) and returns to step 860.

Once vnn and vno are output, they can be saved in local and/or remote memory for future use by the system.

Returning to method 100 in FIG. 1A, at step 180 of the method, the ambulatory ECG device transmits the compressed ECG signal. The compressed ECG signal may be transmitted by the communication interface 250 of the ECG device. The transmission may be wired and/or wireless, directly and/or via a communications network. For example, the compressed ECG signal may be transmitted via a direct interconnection, the Internet, a local area network (“LAN”), a metropolitan area network (“MAN”), a wide area network (“WAN”), a wired or Ethernet connection, a wireless connection, and similar types of communications networks, including combinations thereof. In further embodiments, the communications network 330 can include other communication means, such as Bluetooth, cellular data networks (e.g., 3G, 4G, 5G, LTE, etc.), or the like. In some examples, the ambulatory ECG device may communicate with one or more remote/cloud-based servers, cloud-based services, and/or remote devices via the communications network.

According to an embodiment, the ambulatory ECG device transmits the compressed ECG signal continuously or periodically. The ambulatory ECG device may transmit the compressed ECG signal pursuant to a programmed schedule and/or in response to a command or query. The command or query may be made by a human such as a patient or physician, or the command or query may be made by another local or remote device or system.

Turning now to the steps of method 100 represented in FIG. 1B, at step 190 of the method the transmitted compressed ECG signal is received by a remote device, server, or system. As described or envisioned above, the compressed ECG signal can be transmitted in a variety of different ways. As shown in FIG. 3, the receiving remote server or computer 360 comprises a communication interface 350 that can interact with communications networks and can receive the transmitted signal. Once received, the compressed ECG signal can be stored in memory, and/or can be analyzed by a processor 320 of the remote server or computer 360 to generate a reconstructed ECG signal. The remote server or computer 360 further comprises or is in direct or indirect communication with a user interface 340 that can be utilized to display one or more aspects of a reconstructed ECG signal.

At step 192 of method 100 in FIG. 1B, the system reconstructs the compressed ECG signal to generate a reconstructed approximated ECG signal. The reconstructed approximated ECG signal can be generated according to a variety of different mechanisms. According to an embodiment, processor 320 of remote server or computer 360 comprises a reconstruction algorithm that is configured to generate a reconstructed approximated ECG signal from the received compressed ECG signal.

Referring to FIG. 9, in one embodiment, is a method 900 for generating a reconstructed approximated ECG signal. According to an embodiment, the data is stored in a transmitted HDF5 file 910. At step 920 of the method, the system decodes the metadata stored in the file, using known methods for decoding metadata.

At step 930 of the method, the system reconstructs δ using the vnn and vno values (described in conjunction with FIG. 8; vnn and vno are components of the compressed signal and are thus part of the compressed signal packet).

At step 940 of the method, the system recovers the indices and at step 950 the system recovers the full wavelet coefficients. At step 960 of the method, the system takes the inverse Discrete Wavelet Transform (DWT), which reverses the effect of the Discrete Wavelet Transform (DWT) described above. This results in the reconstructed approximated ECG signal at step 970. Once generated, this reconstructed approximated ECG signal can be utilized immediately, or it can be stored in local and/or remote memory for future use.

Returning to method 100 in FIG. 1B, at step 194 of the method the reconstructed approximated ECG signal is transformed to generate a decompressed ECG signal. The decompressed ECG signal may be generated from the reconstructed approximated ECG signal in a variety of ways. According to an embodiment, the remote server or computer 360 comprises a neural network autoencoder that is trained to generate the original ECG signal from the reconstructed approximated ECG signal.

Referring to FIG. 10, in one embodiment, is a schematic representation of a neural network autoencoder 1000. The fidelity of the compressed and transmitted ECG signal is further improved by transforming the compressed/decompressed signal into a signal that is more similar to the original signal using the neural network autoencoder. The input can be, but is not limited to, the compressed/decompressed signal in time or transformed (e.g., wavelet) and the output is, once the neural network autoencoder is trained, the original or transformed ECG signal with very high fidelity. One benefit of this process is that the entirety of the obtained ECG signal can be compressed, transmitted, received, and reconstructed, in real-time or near real-time. A full ECG signal provides more data than a partial signal as in prior art methods, which can provide life-saving information for patients.

According to an embodiment, the neural network autoencoder is trained with the input being the compressed/decompressed signal in time or transformed (e.g., wavelet) and the output as the original or transformed signal. The algorithm may be a neural network or other trained machine learning model, for example a convolutional neural network (CNN) or a transformer network or other neural network. Thus, according to an embodiment, the system 300 comprises a trained signal reconstruction algorithm that receives the input data (e.g., the compressed/decompressed signal) and outputs the original or transformed ECG signal with very high fidelity. The signal reconstruction algorithm can be trained in a variety of different ways. Training of the signal reconstruction algorithm is further described herein. According to an embodiment as described or otherwise envisioned herein, the algorithm can be trained with the input of the compressed/decompressed wavelet coefficients (before taking the inverse DWT of the wavelet coefficients) and the output can be the original ECG signal transformed using the DWT. The input/output can be transformed using any other filtering techniques.

Referring to FIG. 11, in one embodiment, is a method 1100 for training the neural network autoencoder of the ECG system 300. This method may be performed by the ECG system, or may be performed by another system such as a specialized machine learning model training system.

At step 1110 of the method, the training system receives training data which will be used to train the model. The training data can be any data sufficient to train the model to utilize the described input data to generate the described output. For example, the training data may comprise compressed/decompressed signals together with the original or transformed signals utilized to generate the compressed/decompressed signal. This training data, which could be utilized in a supervised or unsupervised manner, can comprise ECG signals for 100 s or 1000 s of patients and/or procedures, and can be updated with new ECG data at any point. The training data may also comprise other information, such as patient information. This training data may be curated by an expert such as a clinician, or it may be obtained and utilized without curation. The training data may be received from any source. For example, the training data may be received from an electronic medical record database or system, or any other component of the system or a training system. According to an embodiment, the system comprises or is in direct or indirect communication with an ECG signal database which comprises some or all of the training data set.

According to an embodiment, the training system may comprise a data pre-processor or similar component or algorithm configured to process the received training data. For example, the data pre-processor analyzes the training data to remove noise, bias, errors, and other potential issues. The data pre-processor may also analyze the input data to remove low quality data. Many other forms of data pre-processing or data point identification and/or extraction are possible.

At step 1120 of the method, the training system trains the neural network autoencoder of the ECG system 300, using the training data, to reconstruct from input data the original ECG signal. The neural network autoencoder is trained using any method for training such a model. The trained neural network autoencoder is a unique model based on the training data used to train the model. Following training, the system comprises a trained neural network autoencoder. According to an embodiment, the dataset can be trained using any ECG collected.

Following training the neural network autoencoder is a specialized model configured to receive the specialized input (e.g., the compressed/decompressed signal) and generate the very specific output, namely the original ECG signal with very high fidelity.

According to an embodiment, the trained model can be validated through analysis of compression metrics (e.g., percent root deviation and weighted diagnostic distortion) and by clinical review of the ECG signals generated by the model, among other possible methods.

At step 1130 of the method, the trained neural network autoencoder is stored in local and/or remote memory for future use.

Returning to method 100 as depicted in FIG. 1B, at step 196 of the method the system displays some or all of the generated original ECG signal via a user interface of the system, such as user interface 240 and/or user interface 340. The user interface may include one or more devices for enabling communication with a user. The user interface can be any device or system that allows information to be conveyed and/or received, and may include a display, a mouse, and/or a keyboard for receiving user commands. In some embodiments, user interface 240 may include a command line interface or graphical user interface that may be presented to a remote terminal via communication interface 250. The user interface may be located with one or more other components of the system, or may be located remotely from the system and in communication via a wired and/or wireless communications network.

Referring again to FIG. 2 is a schematic representation of an ECG device 200. Device 200 may be any of the devices or systems described or otherwise envisioned herein, and may comprise any of the components described or otherwise envisioned herein. It will be understood that FIG. 2 constitutes, in some respects, an abstraction and that the actual organization of the components of device 200 may be different and more complex than illustrated.

According to an embodiment, device 200 comprises a processor 220 capable of executing instructions stored in memory 230 or storage 260 or otherwise processing data to, for example, perform one or more steps of the method. Processor 220 may be formed of one or multiple modules. Processor 220 may take any suitable form, including but not limited to a microprocessor, microcontroller, multiple microcontrollers, circuitry, field programmable gate array (FPGA), application-specific integrated circuit (ASIC), a single processor, or plural processors.

Memory 230 can take any suitable form, including a non-volatile memory and/or RAM. The memory 230 may include various memories such as, for example L1, L2, or L3 cache or system memory. As such, the memory 230 may include static random access memory (SRAM), dynamic RAM (DRAM), flash memory, read only memory (ROM), or other similar memory devices. The memory can store, among other things, an operating system. The RAM is used by the processor for the temporary storage of data. According to an embodiment, an operating system may contain code which, when executed by the processor, controls operation of one or more components of system 200. It will be apparent that, in embodiments where the processor implements one or more of the functions described herein in hardware, the software described as corresponding to such functionality in other embodiments may be omitted.

User interface 240 may include one or more devices for enabling communication with a user. The user interface can be any device or system that allows information to be conveyed and/or received, and may include a display, a mouse, and/or a keyboard for receiving user commands. In some embodiments, user interface 240 may include a command line interface or graphical user interface that may be presented to a remote terminal via communication interface 250. The user interface may be located with one or more other components of the system, or may be located remotely from the system and in communication via a wired and/or wireless communications network.

Communication interface 250 may include one or more devices for enabling communication with other hardware devices. For example, communication interface 250 may include a network interface card (NIC) configured to communicate according to the Ethernet protocol. Additionally, communication interface 250 may implement a TCP/IP stack for communication according to the TCP/IP protocols. Various alternative or additional hardware or configurations for communication interface 250 will be apparent.

Storage 260 may include one or more machine-readable storage media such as read-only memory (ROM), random-access memory (RAM), magnetic disk storage media, optical storage media, flash-memory devices, or similar storage media. In various embodiments, storage 260 may store instructions for execution by processor 220 or data upon which processor 220 may operate. For example, storage 260 may store an operating system 261 for controlling various operations of system 200.

It will be apparent that various information described as stored in storage 260 may be additionally or alternatively stored in memory 230. In this respect, memory 230 may also be considered to constitute a storage device and storage 260 may be considered a memory. Various other arrangements will be apparent. Further, memory 230 and storage 260 may both be considered to be non-transitory machine-readable media. As used herein, the term non-transitory will be understood to exclude transitory signals but to include all forms of storage, including both volatile and non-volatile memories.

While device 200 is shown as including one of each described component, the various components may be duplicated in various embodiments. For example, processor 220 may include multiple microprocessors that are configured to independently execute the methods described herein or are configured to perform steps or subroutines of the methods described herein such that the multiple processors cooperate to achieve the functionality described herein. Further, where one or more components of device 200 is implemented in a cloud computing system, the various hardware components may belong to separate physical systems. For example, processor 220 may include a first processor in a first server and a second processor in a second server. Many other variations and configurations are possible.

According to an embodiment, device 200 (and/or system 300) may also comprise or be in direct or indirect communication with an electronic medical record system and/or an electronic medical records (EMR) database from which the information about patients, including ECG information, may be obtained or received. For example, EMR database may comprise information about an ECG signal obtained for a patient. According to an embodiment, the electronic medical record system may be a local or remote database and is in direct and/or indirect communication with device 200 (and/or system 300). Thus, according to an embodiment, the device or system comprises an electronic medical record database or system.

According to an embodiment, storage 260 of device 200 may store one or more algorithms, modules, and/or instructions to carry out one or more functions or steps of the methods described or otherwise envisioned herein. For example, storage 260 may comprise, among other instructions or data, ECG signal instructions 262, compression instructions 263, and/or transmission instructions 264.

According to an embodiment, ECG signal instructions 262 direct the system to obtain ECG data from a patient. The ECG device may obtain an ECG signal using known methods for obtaining ECG signals. The signal may be obtained continuously or periodically as determined by the settings or parameters of the ECG device or system. When the ECG device is an ambulatory device, the ECG signal may be obtained while the subject is ambulatory. Thus, according to an embodiment, “ambulatory” can mean that the subject is utilizing a mobile ECG device outside a clinical setting, rather than using a stationary or mobile ECG device within a clinical setting. Once obtained, the ECG signal may be utilized immediately and/or it may be saved in memory for future analysis.

According to an embodiment, compression instructions 263 direct the system to compress the obtained ECG signal as described or otherwise envisioned herein, including but not limited to the method depicted in FIG. 1A. Once compressed, the ECG signal may be utilized immediately and/or it may be saved in memory for future analysis.

According to an embodiment, transmission instructions 264 direct the system to transmit the compressed ECG signal. The compressed ECG signal may be transmitted by the communication interface 250 of the ECG device. The transmission may be wired and/or wireless, directly and/or via a communications network. According to an embodiment, the ambulatory ECG device transmits the compressed ECG signal continuously or periodically. The ambulatory ECG device may transmit the compressed ECG signal pursuant to a programmed schedule and/or in response to a command or query. The command or query may be made by a human such as a patient or physician, or the command or query may be made by another local or remote device or system.

Referring to FIG. 3, in one embodiment, is an ECG system 300 is provided. Referring to an embodiment of an ECG system 300 as depicted in FIG. 3, for example, the system comprises one or more of an ECG device 200, which according to an embodiment is an ambulatory ECG device, which comprises one or more ECG leads 270 configured to obtain an ECG signal from a subject 310. System 300 further comprises a remote server or computer 360, which receives a compressed ECG signal from the ECG device 200. For example, the compressed ECG signal can be transmitted by the communication interface 250 of the ECG device to the communication interface 350 of the remote server or computer 360, either directly or through a communications network 330. Once received, the compressed ECG signal can be stored in memory, and/or can be analyzed by a processor 320 of the remote server or computer 360 to generate a reconstructed ECG signal. The remote server or computer 360 further comprises or is in direct or indirect communication with a user interface 340 that can be utilized to display one or more aspects of a reconstructed ECG signal.

According to an embodiment, remote server or computer 360 further comprises storage or memory 360. Storage 260 may include one or more machine-readable storage media such as read-only memory (ROM), random-access memory (RAM), magnetic disk storage media, optical storage media, flash-memory devices, or similar storage media. In various embodiments, storage 360 may store instructions for execution by processor 320 or data upon which processor 320 may operate. For example, storage 360 may store an operating system 361 for controlling various operations of system 300. Storage 360 may be considered to be non-transitory machine-readable media. As used herein, the term non-transitory will be understood to exclude transitory signals but to include all forms of storage, including both volatile and non-volatile memories.

According to an embodiment, storage 360 of system 300 may store one or more algorithms, modules, and/or instructions to carry out one or more functions or steps of the methods described or otherwise envisioned herein. For example, storage 360 may comprise, among other instructions or data, trained neural network encoder 363, training instructions 263, and/or reporting instructions 264.

According to an embodiment, the trained neural network encoder 363 of the system is trained to analyze the received input, including but not limited to a compressed/decompressed signal, and generate the very specific output, namely the original ECG signal with very high fidelity. The trained neural network encoder can be any model that can be trained to utilize the input to generate the output, as described or otherwise envisioned herein. For example, the neural network encoder can be a neural network or other trained machine learning model, for example a convolutional neural network (CNN) or a transformer network or other neural network. Thus, according to an embodiment, the ECG system 300 comprises a trained neural network encoder that receives the input data and outputs at least the original ECG signal.

According to an embodiment, training instructions 263 direct the system to train a neural network encoder 363 of the ECG system 300. The instructions direct the system to retrieve, obtain, or receive training data. The training data can be any data sufficient to train the model to utilize the described input data to generate the described output. For example, the training data may comprise compressed/decompressed signals together with the original or transformed signals utilized to generate the compressed/decompressed signal. This training data, which could be utilized in a supervised or unsupervised manner, can comprise ECG signals for 100 s or 1000 s of patients and/or procedures, and can be updated with new ECG data at any point. The training data may also comprise other information, such as patient information. This training data may be curated by an expert such as a clinician, or it may be obtained and utilized without curation. The training data may be received from any source. For example, the training data may be received from an electronic medical record database or system, or any other component of the system or a training system. According to an embodiment, the system comprises or is in direct or indirect communication with an ECG signal database which comprises some or all of the training data set. The training instructions 363 further direct the system to train the model using the obtained training data. The model can be trained using a variety of different training methods. The training instructions 363 further direct the system to store the trained model for future use.

According to an embodiment, reporting instructions 364 direct the system to provide the output of the system—such as the regenerated ECG signal—to a patient, clinician, or other device or system. The provided output can be any of the information as described or otherwise envisioned herein. The system may provide the information to a user via any mechanism, including but not limited to a visual display, an audible notification, a page, or any other method of notification. The information may be communicated by wired and/or wireless communication to another device. For example, the system may communicate the information to a monitor, screen, mobile phone, computer, laptop, wearable device, and/or any other device configured to allow display and/or other communication of the information. According to an embodiment, the system displays some or all of the generated original ECG signal via a user interface of the system, such as user interface 240 and/or user interface 340.

According to an embodiment, the ECG system 300 is configured to process many thousands or millions of datapoints in the input data used to train neural network encoder 363. For example, generating a functional and skilled trained neural network encoder from a corpus of training data requires processing of millions of datapoints from input data and generated features. This can require millions or billions of calculations to generate a novel trained neural network encoder from those millions of datapoints and millions or billions of calculations. As a result, each trained neural network encoder is novel and distinct based on the input data and parameters of the model, and thus improves the functioning of the system. Generating a functional and skilled trained neural network encoder comprises a process with a volume of calculation and analysis that a human brain cannot accomplish in a lifetime, or multiple lifetimes.

### Examples

The following is provided as an example of the methods and systems configured to generate a high-fidelity ECG signal from a transmitted compressed ECG signal, as described or otherwise envisioned herein. Accordingly, it is understood that these are non-limiting examples, and that the methods and systems described or otherwise envisioned herein can be utilized for a variety of purposes, applications, and situations.

Referring to FIG. 12, in one embodiment, is a graph 1200 of a single heartbeat from an ECG signal. As described or otherwise envisioned herein, the original signal was compressed and then reconstruction to generate a high-fidelity ECG signal from the compressed ECG signal, pursuant to the method shown in FIGS. 1A and 1B. Shown in FIG. 12 are the original signal, the compressed signal, and the signal after reconstruction to generate a high-fidelity ECG signal. As shown in FIG. 12, the reconstructed signal is significantly more similar to the original signal than the compressed ECG signal, demonstrating that the methods and systems described or otherwise envisioned herein provide a significant improvement in the compression, transmission, and subsequent analysis of the original ECG signal.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

What is claimed is:

1. A method for compressing and transmitting an ECG signal by an ambulatory electrocardiogram (ECG) device, comprising:
obtaining, by the ambulatory ECG device, an ECG signal from a subject;
determining, by a processor of the ambulatory ECG device, whether the obtained ECG signal comprises ECG beat locations;
pre-processing, by the processor, the obtained ECG signal with a de-trending analysis to generate a pre-processed ECG signal if the obtained ECG signal is determined to comprise ECG beat locations, or pre-processing, by the processor, the obtained ECG signal with a bandpass filter to generate a pre-processed ECG signal if the obtained ECG signal is determined to not comprise ECG beat locations;
approximating, by the processor, the pre-processed ECG signal to generate an approximated ECG signal;
quantizing, by the processor, the approximated ECG signal to generate a quantized ECG signal;
generating, by a compression algorithm of the processor, a compressed ECG signal from the quantized ECG signal;
transmitting, by the ambulatory ECG device, the compressed ECG signal.

2. The method of claim 1, further comprising:
receiving, the transmitted compressed ECG signal;
reconstructing, by a reconstruction algorithm, the compressed ECG signal to generate a reconstructed approximated ECG signal;
transforming, by a trained neural network, the reconstructed approximated ECG signal to generate a decompressed ECG signal.

3. The method of claim 2, further comprising the step of displaying at least a portion of the decompressed ECG signal.

4. The method of claim 2, wherein the transmitted compressed ECG signal is received, reconstructed, and analyzed by a server remote from the ambulatory ECG device.

5. The method of claim 1, wherein approximating the pre-processed ECG signal to generate an approximated ECG signal comprises: (i) a wavelet transform; (ii) a selection of largest wavelet coefficients; and (iii) a tolerance determination.

6. The method of claim 5, wherein a one-second windowed ECG signal around each beat in at least a portion of the obtained ECG signal is utilized to determine the tolerance.

7. The method of claim 1, wherein quantizing the approximated ECG signal to generate a quantized ECG signal comprises quantizing wavelet coefficients of the ECG signal using a quantization parameter (A).

8. The method of claim 1, wherein generating a compressed ECG signal from the quantized ECG signal comprises saving the compressed ECG signal as an HDF5 file.

9. The method of claim 1, wherein the compressed ECG signal further comprises encoded metadata.

10. An ambulatory electrocardiogram (ECG) device, comprising:
one or more ECG leads configured to obtain an ECG signal from a subject;
a processor configured to: (i) determine whether the obtained ECG signal comprises ECG beat locations; (ii) pre-process the obtained ECG signal with a de-trending analysis to generate a pre-processed ECG signal if the obtained ECG signal is determined to comprise ECG beat locations, or pre-process the obtained ECG signal with a bandpass filter to generate a pre-processed ECG signal if the obtained ECG signal is determined to not comprise ECG beat locations; (iii) approximate, by the processor, the pre-processed ECG signal to generate an approximated ECG signal; (iv) quantize the approximated ECG signal to generate a quantized ECG signal; and (v) generate a compressed ECG signal from the quantized ECG signal; and
a communications interface configured to transmit the compressed ECG signal.

11. The device of claim 10, wherein approximating the pre-processed ECG signal to generate an approximated ECG signal comprises: (i) a wavelet transform; (ii) a selection of largest wavelet coefficients; and (iii) a tolerance determination.

12. The device of claim 11, wherein a one-second windowed ECG signal around each beat in at least a portion of the obtained ECG signal is utilized to determine the tolerance.

13. The device of claim 10, wherein generating a compressed ECG signal from the quantized ECG signal comprises saving the compressed ECG signal as an HDF5 file.

14. An electrocardiogram (ECG) system, comprising:
an ambulatory ECG device comprising:
one or more ECG leads configured to obtain an ECG signal from a subject;
a processor configured to: (i) determine whether the obtained ECG signal comprises ECG beat locations; (ii) pre-process the obtained ECG signal with a de-trending analysis to generate a pre-processed ECG signal if the obtained ECG signal is determined to comprise ECG beat locations, or pre-process the obtained ECG signal with a bandpass filter to generate a pre-processed ECG signal if the obtained ECG signal is determined to not comprise ECG beat locations; (iii) approximate, by the processor, the pre-processed ECG signal to generate an approximated ECG signal; (iv) quantize the approximated ECG signal to generate a quantized ECG signal; and (v) generate a compressed ECG signal from the quantized ECG signal;
a communications interface configured to transmit the compressed ECG signal; and
a remote server comprising:
a communications interface configured to receive the transmitted compressed ECG signal
a processor configured to: (i) reconstruct the compressed ECG signal to generate a reconstructed approximated ECG signal; and (ii) transform, by a trained neural network, the reconstructed approximated ECG signal to generate a decompressed ECG signal.

15. The system of claim 14, wherein the remote server further comprises a user interface configured to display at least a portion of the decompressed ECG signal.
